(19) United States
(12) Patent Application Publication
(10) Pub. No.: US 2021/0292265 A1
(43) Pub. Date: Sep. 23, 2021

(54) CHALCONES AND DERIVATIVES FOR USE IN MEDICAMENTS AND NUTRACEUTICALS

(71) Applicant: HLXTH B.V., Oegstgeest (NL)

(72) Inventor: Stefan Frederik Franciscus VERLINDEN, Oegstgeest (NL)

(73) Assignee: HLXTH B.V., Oegstgeest (NL)

(21) Appl. No.: 17/261,129

(22) PCT Filed: Jul. 24, 2019
(86) PCT No.: PCT/NL2019/050479

§ 371 Date: Jan. 18, 2021

(30) Foreign Application Priority Data
Jul. 24, 2018 (EP) 18185325.0

(51) Int. Cl. C07C 49/835; C07C 49/84

(52) U.S. Cl. CPC C07C 49/835; A23L 33/10; C07C 49/84

(57) ABSTRACT

Provided herein are means and methods for inhibiting processes and/or facilitating processes in cells by means of a compound of structural formula I

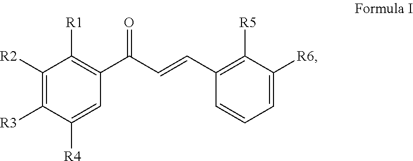

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH. The compound can be used among others in the prophylactic or curative treatment of an elevated blood interleukin-1β level and/or the treatment of low grade inflammation in an animal subject in need thereof. Also provided is a method for increasing longevity or increasing the health span in a non-diseased animal subject, the method including administering to the animal subject an effective amount of a hydroxychalcone of formula I as indicated herein. Also provided are food and food supplements including a compound of formula I as indicated herein.

The invention relates to chalcones and derivatives thereof for use in the prophylactic or curative treatment of an elevated blood interleukin-1β level or an elevated blood interleukin-18 level in an animal subject in need thereof and/or the treatment of low grade inflammation in an animal subject in need thereof. The invention also relates to a method for increasing longevity in a non-diseased animal subject, the method comprising administering to the animal subject an effective amount of a particular hydroxychalcone as described herein. The invention also relates to means and methods for inhibiting caspase-1, interleukin-16 and/or IL-18 over-expression, inhibiting mTOR and/or inhibiting NLRP-3.

Human aging is characterized by a chronic, low-grade inflammation. This phenomenon has been termed “inflammaging.” One source of inflammaging could be the damaged macromolecules and cells (self-debris) that accumulate with age due to increased production and/or inadequate elimination. Self-debris released as a consequence of cell/organelle injury can mimic bacterial products and function as endogenous “damage”-associated molecular patterns that activate innate immunity.

Damaged cellular and organelle components, free radicals from oxidative stress, metabolites such as extracellular ATP, fatty acids, urate crystals, ceramides, cardiolipin, amyloid, succinate, per-oxidized lipids, advanced glycation end-products, altered N-glycans (3), and HMGB1 are recognized by a network of sensors (including NLRP3 inflammasome) as “danger” signals and initiate immune reactions that are necessary for physiological repair. However, as damage accumulates, the danger responses can become chronic and hence maladaptive.

The large variety of the stimuli fueling inflammaging apparently converge on few basic mechanisms and pathways such as activation of NF-κB and NLRP3 inflammasome, responsible for the production of inflammatory molecules. Chronic low grade inflammation (LGI) is a highly significant risk factor for both morbidity and mortality in the elderly people, as many age-related diseases share an inflammatory pathogenesis (Franceschi and Campisi, J. Gerontol A Biol Sci Med Sci 2014: 69: S4-S9) 2014). Franceschi coined the term “inflammaging” at the turn of the millennium as part of the spectrum of immunosenescence. Inflammaging denotes an upregulation of the inflammatory response that occurs with age, resulting in a low-grade chronic systemic proinflammatory state further referred to as “chronic low grade inflammation” or LGI. It is characterized by raised levels of proinflammatory cytokines interleukin-1 (IL-1), interleukin-6 (IL-6) and tumour necrosis factor (TNF); all of which have been shown to rise with age (Vasto S et al 2007 Mech Ageing Dev. 2007; Vol 2:83-91. doi: 10.1016/j.mad.2006.11.015) and to be involved in the pathogenesis of most age-associated diseases (De Martinis M 2006 Exp Mol Pathol. 2006; Vol 2:219-227. doi: 10.1016/j.yexmp.2005.11.004). The level of the proinflammatory cytokines interleukin-1 (IL-1), interleukin-6 (IL-6) and tumour necrosis factor (TNF) is typically chronically elevated to around 2- to 3-fold (Antonelli M, Kushner I. It's time to redefine inflammation. FASEB J. 2017 May; 31(5):1787-1791).

An inflammatory response is beneficial as an acute, transient reaction to harmful conditions. It facilitates the defense, repair, turnover and adaptation of many tissues. However, LGI is detrimental for many tissues and for normal functions. Major health complications associated with LGI in the elderly include mental health and wellbeing, metabolic abnormalities and infections (Calder et al, Ageing Research Reviews, 2017, 40: 95-119). The authors provide an overview of the evidence that exists in the elderly for omega-3 fatty acid, probiotic, prebiotic, antioxidant and polyphenol interventions as a means to influence LGI. Slowing, controlling or reversing LGI is an important way to prevent, or reduce the severity of, age-related functional decline and the onset of conditions affecting health and wellbeing (Calder et al 2017, supra); Calder et al further provide evidence to support specific dietary interventions as a strategy to control LGI; and state that a continued research focus on this field is warranted.

Chalcones (or chalcone derivatives) are compounds having a basic C6-C3-C6 arrangement in which the middle three carbon atoms do not form a closed ring. Various chalcones are found in plants where they are, among others, precursors in the synthesis of pigment in plant. Many of the compounds have anti-oxidant effect and provide some protection from harmful UV irradiation (Woo W. S.; Methodology of natural product chemistry (Seoul National University Publishing), pp 131-137).

Chalcones are abundant in plants of the genus Corepsis. Chalcones of natural origin 2′6′-dihydroxy-4-methoxychalcone, carthamin, and butein are identified from plants such as cinnamon, red pepper and carthamus flower. Dihydrochalcone is contained in certain species of the genus Rosaceae and Rhododendron, and phloridzin is one of the components in apple tree foliage (Hunter, M.D.; Phytochemistry (Oxford) 34, pp 1251-1254, 1993). Nowadays many new and known chalcones are produced synthetically in the lab. Some chalcones are known to inhibit glucose transport and growth of various cells including cancer.

In the present invention it was found that particular chalcones or derivatives thereof are potent inhibitors of the activity of the protein Mechanistic Target Of Rapamycin (mTOR). In the present invention it was also found that some chalcones or derivatives thereof are potent inhibitors of the activity of the protein NLR Family, Pyrin Domain Containing 3 (NLRP-3). It was also found that some chalcones or derivatives thereof inhibit interleukin-1β (IL-1β or IL-1beta) expression in IL-1β expressing cells. It was also found that some chalcones or derivatives thereof inhibit interleukin-18 (IL-18) expression in IL-18 expressing cells. It was also found that some chalcones or derivatives thereof inhibit the formation of caspase-1 in pro-caspase-1 expressing cells. Inactive caspase-1 is produced as a zymogen that can then be cleaved into 20 kDa (p20) and 10 kDa (p10) subunits that become part of the active enzyme. Inactive caspase-1 is also referred to as pro-caspase-1. Active Caspase 1 contains two heterodimers of p20 and p10. It contains a catalytic domain with an active site that spans both the p20 and p10 subunits, as well as a noncatalytic Caspase Activation and Recruitment Domain (CARD). Active Caspase 1 is also referred to as caspase-1. Inactive caspase-1 is typically activated when it is assembled into an inflammasome.

IL-1β is produced from IL-1β precursor that is translated from mRNA in the cell. The IL-1β precursor is cleaved by cytosolic caspase 1 (interleukin 1 beta convertase) to form mature IL-1β. When herein reference is made to IL-1β expression the reference is to the production of the mature form of IL-1β from the precursor. IL-18 is synthesized as an inactive 24 kDa propeptide which is activated by proteolytic cleavage by caspase-1 in the NLRP3 inflammasome. Cleavage generates the biologically functional 18 kDa IL-18 molecule. IL-1β and IL-18 are typically excreted from the producing cell. When herein reference is made to IL-18 expression the reference is to the production of the active form of IL-18 from the inactive 24 kDa propeptide.

The mentioned interleukins are associated with chronic low grade inflammation in humans (see Calder et al 2017: Ageing research Reviews 40: 95-119). Inflammation in a tumor microenvironment mediated by interleukin 16 is hypothesized to have a major role in cancer invasiveness, progression, and metastases. Various effects were noted in clinical trials with the anti-interleukin-1β antibody canakinumab. Ridker et al performed an analysis in the Canakinumab Anti-inflammatory Thrombosis Outcomes Study (CANTOS), a randomized trial of the role of interleukin-1β inhibition in atherosclerosis, with the aim of establishing whether inhibition of a major product of the Nod-like receptor protein 3 (NLRP3) inflammasome with canakinumab might alter cancer incidence (Ridker et al. Lancet. 2017 Oct. 21; 390(10105):1833-1842). On the basis of the results Ridker et al hypothesized that anti-inflammatory therapy with canakinumab targeting the interleukin-1β innate immunity pathway could significantly reduce incident lung cancer and lung cancer mortality. In another paper on the same study it was noted that the canakinumab IL-1β antibody exhibits a dose effect on the incidence of gout and osteoarthritis in the treated patients (Ridker et al. N Engl J Med. 2017 Sep. 21; 377(12):1119-1131. doi: 10.1056/NEJMoa1707914). The authors of this paper conclude that particular dosing of the IL-1β antibody significantly reduced the rate of recurrent cardiovascular events than placebo, independent of lipid-level lowering.

### SUMMARY OF THE INVENTION

The invention provides a pharmaceutical composition comprising a hydroxychalcone of formula I and a pharmaceutical carrier or excipient

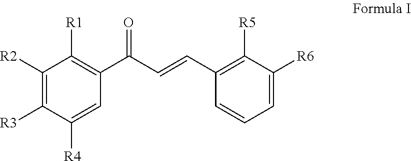

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH; and wherein the hydroxychalcone is not a hydroxychalcone wherein R1, R3 and R5 is OH and R2, R4 and R6 is H.

Also provided is a hydroxychalcone of formula I

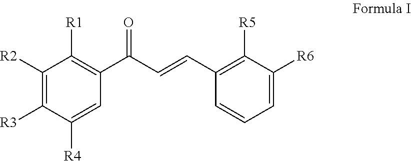

wherein
R1 is CH₃; R2 is H; R3 is OH; R4 is H; R5 is CH₃; R6 is H; or
R1 is OH; R2 is H; R3 is CH₃; R4 is H; R5 is CH₃; R6 is H; or
R1 is H; R2 is CH₃; R3 is H; R4 is OH; R5 is CH₃; R6 is H; or

### R1 is CH3; R2 is H; R3 is OH; R4 is H; R5 is OH; R6 is H; or

### R1 is OCH3; R2 is H; R3 is OH; R4 is H; R5 is OH; RG is H; or

### R1 is OH; R2 is H; R3 is OH; R4 is H; R5 is CH₃; R6 is H; or

### R1 is OH; R2 is H; R3 is OH; R4 is H; R5 is OH; R6 is OH.

Further provided is a method of inhibiting a Mechanistic Target Of Rapamycin (mTOR) protein and/or NLRP-3 in a cell, the method comprising contacting the cell with a hydroxychalcone of formula I referred to herein.

The invention further provides a method of treatment of an animal subject that has a disease associated with caspase-1, interleukin-1β and/or IL-18 over-expression, or has an increased risk of developing said disease, the method comprising administering the compound of formula I to the animal subject in need thereof.

Also provided is a compound of formula I for use in the treatment of an animal subject that has a disease associated with caspase-1, interleukin-1β and/or IL-18 over-expression, or has an increased risk of developing said disease.

Further provided is the use of a compound of formula I for the preparation of a medicament; a food or a food supplement for the treatment of an animal subject that has a disease associated with caspase-1, interleukin-1β and/or IL-18 over-expression, or has an increased risk of developing said disease.

Also provided is a method of stimulating autophagy and/or phagocytosis in a cell, the method comprising contacting the cell with a hydroxychalcone of formula I referred to herein.

Also provided is a method of inhibiting caspase-1, interleukin-1β and/or IL-18 production by a cell, the method comprising contacting the cell with a hydroxychalcone of formula I referred to herein. The cell is preferably a hematopoietic cell of the monocytic lineage, such as (but not limited to) a monocyte, a macrophage, a Kupfer cell and/or a microglia cell. The cell is preferably a macrophage. Inhibition of caspase-1, interleukin-1β and/or IL-18 production by a cell, refers to the inhibition of the formation of mature/activated forms of the respective proteins.

Further provided is a food product or a food supplement comprising a hydroxychalcone of formula I referred to herein. The food supplement preferably comprises 20-2000 mg of the hydroxychalcone of formula I referred to herein.

Also provided is a method for increasing longevity in a non-diseased animal subject, the method comprising administering to the animal subject an effective amount of a hydroxychalcone of formula I

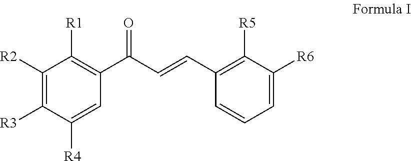

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH.

Also provided is a hydroxychalcone of formula I

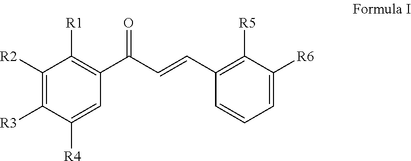

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH for use in increasing longevity and/or increasing the health span in a non-diseased animal subject.

Also provided is a method of treatment of an animal subject that has an elevated blood interleukin-1β level and/or has chronic low grade inflammation, the method comprising administering the hydroxychalcone of formula I to the animal subject in need thereof.

Also provided is a compound of formula I for use in the treatment of an animal subject that has an elevated blood interleukin-1β level and/or has chronic low grade inflammation.

Further provided is the use of a compound of formula I for the preparation of a medicament; a food or a food supplement for the treatment of an animal subject that an elevated blood interleukin-1β level and/or has chronic low grade inflammation.

A compound of formula I for use in the treatment of an animal subject with a disease characterized in that symptoms of the disease are ameliorated by inhibiting a Mechanistic Target Of Rapamycin (mTOR) protein and/or NLRP-3 protein in a cell of said animal subject.

Also provided is a compound of formula I for use in the treatment of an animal subject with a disease characterized in that symptoms of the disease are ameliorated by stimulating autophagy and/or phagocytosis in a cell of said animal subject.

Provided is also a compound of formula I for use in the treatment of a disease that benefits from inhibiting caspase-1, interleukin-1β and/or IL-18 production by a cell of said animal subject.

Also provided is a food or a food supplement comprising a compound of formula I.

The compound of formula I is preferably administered in a therapeutically effective amount to (i) reduce levels of interleukin-1β and/or IL-18 in plasma of the subject, (ii) inhibit NLRP3 inflammasome-mediated IL-1β expression in macrophages or dendritic cells of the subject (i.e. production of mature or (iii) inhibit caspase-1 in macrophages or dendritic cells of the subject (i.e. the production of active caspase-1). The compound of formula I is preferably administered in a therapeutically effective amount to reduce phospho-PP70-S6K macrophages of the subject. When cells of the subject are indicated, it is preferred that the cells are cells of the blood.

### DETAILED DESCRIPTION OF THE INVENTION

NLRPs and IPAF subfamilies are involved in the formation of the inflammasome. The best characterized inflammasome is the NLRP3 inflammasome. NLRP1, NLRP3 and NLRC4 are subsets of the NLR family and have two common features: the first is a nucleotide-binding domain (NPD) which is bound by ribonucleotide-phosphates (rNTP) and is important for self-oligomerization. The second is a C-terminus leucine-rich repeat (LRR), which serves as a ligand-recognition domain for other receptors (e.g. TLR) or microbial ligands. The NLRP-3 inflammasome is so-called because off the NLRP-3 protein in the complex. The NLRP-3 inflammasome is associated with onset and progression of various diseases including auto-immune and auto-inflammatory diseases. Several NLRP-3 inflammasome inhibitors have been described, some of which show promise in the clinic. In the absence of an activating signal NLRP-3 is kept in an inactive state complexed with HSP90 and SGT1 in the cytoplasm. NLRP-3 inflammasome detects danger signals such as crystalline uric acid and extracellular ATP released by damaged cells. These signals cause a release of NLRP-3 from HSP90 and SGT1. Caspase-1 formed by the activated NLRP-3 inflammasome complex in turn activates the inflammatory cytokine, IL-1β (for review see Shoa et al, frontiers in Pharmacology 2015, Vol. 6, 1-9). Various NRLP3 inhibitors are known. The small molecule, inhibitor MCC950 inhibits the canonical and non-canonical activation of the NLRP-3 inflammasome. It inhibits the secretion of IL-1β and IL-18 (Coll et al. 2015, Nat. Med 21, 248-255). The compound BHB also reduces IL-1β and IL-18 production (Youm et al 2015 Nat Med 21, 263-369).

Inflammaging refers to a low-grade pro-inflammatory phenotype which accompanies aging in mammals. The aging process is associated with a decline in autophagy capacity which impairs cellular housekeeping. This leads to protein aggregation and accumulation of dysfunctional mitochondria which provoke reactive oxygen species (ROS) production and oxidative stress. Recent studies have indicated that the ROS production induced by damaged mitochondria can stimulate intracellular danger-sensing multiprotein platforms called inflammasomes. NLRP-3 can be activated by various danger signals, e.g. ROS, cathepsin B released from destabilized lysosomes and aggregated proteins, all of which evoke cellular stress and are involved in the aging process. NLRP-3 activation is also enhanced in aging. NLRP-3 activates inflammatory caspases, mostly caspase-1, which cleave the inactive precursors of IL-1β and IL-18 and stimulate their secretion. Consequently, these cytokines provoke low grade inflammatory responses which accelerates the aging process. A compound of formula I stimulates autophagic capacity with aging and at least in part negates the effects of ageing. Consistent with this is that systemic low-grade inflammation promotes age-related degenerative changes, the deficient NLRP-3 inflammasome-mediated caspase-1 activity improved glycemic control and attenuated bone loss and thymic demise. Notably, IL-1β mediated only NLRP-3 inflammasome-dependent improvement in cognitive function and motor performance in aged mice. These studies reveal NLRP-3 inflammasome as an upstream target that controls age-related inflammation and offer an innovative therapeutic strategy to lower NLRP-3 activity to delay multiple age-related chronic diseases (Youm Y H et al. Cell Metab. 2013 Oct. 1; 18(4):519-32).

NLRP-3 is known under a number of different names such as: NLR Family, Pyrin Domain Containing 3; Cryopyrin; Cold-Induced Autoinflammatory Syndrome 1 Protein; PYRIN-Containing APAF1-Like Protein 1; Caterpiller Protein 1.1; C1orf7; CLR1.1; PYPAF1; CIAS1; NALP3; Nucleotide-Binding Oligomerization Domain, Leucine Rich Repeat And Pyrin Domain Containing 3; NACHT Domain-, Leucine-Rich Repeat-, And PYD-Containing Protein 3; Leucine Rich Repeat And Pyrin Domain Containing 3; Angiotensin/Vasopressin Receptor AII/AVP-Like; Nucleotide-Binding Oligomerization Domain; Cold Autoinflammatory Syndrome 1 Protein 3; NACHT, LRR And PYD Containing Protein 3; Cold Autoinflammatory Syndrome 1; AGTAVPRL; FCAS1; FCAS; AVP; AII; FCU; and MWS. External ids are HGNC: 16400; Entrez Gene: 114548; Ensembl: ENSG00000162711; OMIM: 606416; and UniProtKB: Q96P20.

Isoliquiritigenin (ILG) is a simple chalcone-type flavonoid. It can be isolated from licorice root (Glycyrrhiza uralensis). It exhibits anti-oxidant, anti-inflammatory, and anti-tumor activities. It is reported to be a potent inhibitor of NLRP-3 (Honda H. et al., 2014. J Leukoc Biol. 96(6):1087-100). Another inhibitor of NLRP-3 is resveratrol (Shoa et al, frontiers in Pharmacology 2015, Vol. 6, 1-9).

The structural formula of ILG is

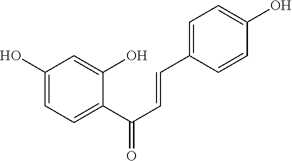 [SMILES: O=C(/C=C/c1ccc(O)cc1)c1ccc(O)cc1O]

The structural formula of resveratrol is

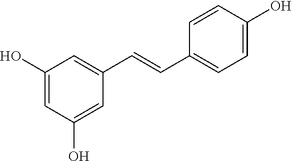 [SMILES: Oc1ccc(/C=C/c2cc(O)cc(O)c2)cc1]

The inventors have discovered that the presence and positioning of radicals on the structural backbone affects the activity of the resulting compounds. The inventors have found a new group of compounds (the compounds of structural formula I) with improved activity.

The compound of formula I has the general formula

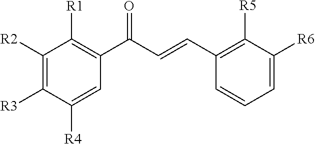

wherein R1, R2, R3, R4, R5 and R6 are each independently H, OH, CH3, OCH3, monosaccharide, an oligosaccharide or Cl; with the proviso that at least two of R1-R6 are H; and at least one other of R1-R6 is OH;
When herein reference is made to a compound of formula I, the reference includes the particular indications of for the radicals R1-R6. Groups not indicated in the structure are preferably H. However, at positions 4, 5, 6 and 6′ it is preferred that the radical is a radical indicated for that position in FIG. 6. It is preferred that the compound is a compound of FIG. 6. In a preferred embodiment it is preferred that the radical at positions 4, 5, 6 and 6′ is H.

In a preferred embodiment R1, R2, R3, R4, R5 and R6 are each independently H, OH, CH3, OCH3, a monosaccharide, or an oligosaccharide, with the proviso that at least one of R1, R3 and R6 is OH, and at least two of R1-R6 are H. In a preferred embodiment at least two of R1, R3 and R6 or OH. In a preferred embodiment R5=CH3 or OCH3, preferably CH3. The compound is preferably a chalcone of FIG. 3. In a preferred embodiment the chalcone of FIG. 6, is a chalcone wherein R6 (or position 3)=H. In a preferred embodiment position 4 is H.

The invention also provides a hydroxychalcone of formula I

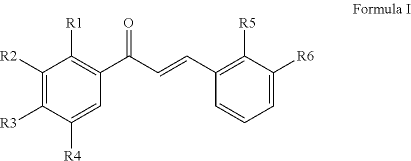

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH; for use in the prophylactic or curative treatment of an elevated blood interleukin-1β level or an elevated blood interleukin-18 level and/or the treatment of low grade inflammation in an animal subject in need thereof. R1, R2, R3, R4, R5 and R6 can each independently H, OH, CH3, or OCH3. In an embodiment R1-R5 are each independently H, OH, CH3, OCH3 and R6 is H, with the proviso that at least one of R1-R5 is H. In some embodiments R1 is CH₃; OH; OCH3 or H; R2 is H or CH3; R3 is OH; CH3 or H; R4 is OH or H; R5 is CH3 or OH and R6 is H, with the proviso that at least two of R1-R5 is H. The hydroxychalcone for use indicated herein is preferably a hydroxychalcone wherein
R1 is CH₃; R2 is H; R3 is OH; RA is H; R5 is CH₃; R6 is H; or
R1 is OH; R2 is H; R3 is CH₃; R4 is H; R5 is CH₃; R6 is H; or
R1 is H; R2 is CH₃; R3 is H; R4 is OH; R5 is CH₃; R6 is H; or

### R1 is CH₃; R2 is H; R3 is OH; R4 is H; R5 is OH; R6 is H; or

### R1 is OCH3; R2 is H; R3 is OH; R4 is H; R5 is OH; R6 is H; or

### R1 is OH; R2 is H; R3 is OH; R4 is H; R5 is CH₃; R6 is H; or

### R1 is OH; R2 is H; R3 is OH; R4 is H; R5 is OH; R6 is OH; or

### R1 is OH R2 is H; R3 is OH; R4 is H; R5 is OH; R6 is H.

In one embodiment the hydroxychalcone in a method of treatment as indicated herein or a four use in a treatment as indicated herein is a hydroxychalcone of formula I of table 6 column 1 with the R1-R6 radicals as indicated for the compound. In a preferred embodiment the hydroxychalcone is the hydroxychalcone of formula I wherein R1 is OH; R2 is H; R3 is OH; R4 is H; R5 is OH; R6 is H.
The invention also provides a hydroxychalcone of formula I as depicted in table 6 column 1, wherein R1-R6 are as indicated for the respective compounds. In a preferred embodiment R1-R6 are:
R1 is CH₃; R2 is H; R3 is OH; R4 is H; R5 is CH₃; R6 is H; or
R1 is OH; R2 is H; R3 is CH₃; R4 is H; R5 is CH₃; R6 is H; or
R1 is H; R2 is CH₃; R3 is H; R4 is OH; R5 is CH₃; R6 is H; or

### R1 is CH₃; R2 is H; R3 is OH; R4 is H; R5 is OH; R6 is H; or

### R1 is OCH3; R2 is H; R3 is OH; R4 is H; R5 is OH; R6 is H; or

### R1 is OH; R2 is H; R3 is OH; R4 is H; R5 is CH₃; R6 is H; or

### R1 is OH; R2 is H; R3 is OH; R4 is H; R5 is OH; R6 is OH.

Compounds of the invention are typically well tolerated, more active than ILG or resveratrol. Another advantage of a compound of the invention is that they are easily produced at affordable prices. This allows among others the economic production of nutraceuticals, foods and food supplements. One of the preferred compounds, 2, 2′, 4′ trihydroxychalcone is commercially available from abcr GmbH, Im Schlehert 10, 76187 Karlsruhe Deutschland (cat number AB151762). Chalcones can be prepared in various manners, for instance by an aldol condensation between benzaldehyde and acetophenone in the presence of sodium hydroxide as a catalyst. By varying the side groups (replacing H) on the benzaldehyde and/or acetophenone, different chalcones can be created. For instance isoliquiritigenin can be formed from 4-hydroxybenzaldehyde and 1-(2,4-dihydroxyphenyl)ethanone. Similarly 2,2′,4′-trihydroxychalcone can be formed from 2-hydroxybenzaldehyde and 1-(2,4-dihydroxyphenyl)ethanone. By combining 2-hydroxybenzaldehyde and 2′-hydroxy-4′-methoxyactetophenone, 2′,4-dihydroxy-4′-methoxychalcone chalcone can be formed. Synthesis of isoliquiritin (4-[(1E)-3-(2,4-dihydroxyphenyl)-3-oxoprop-1-en-1-yl]phenyl beta-D-glucopyranoside) was accomplished starting from 4-hydroxybenzaldehyde and 2,4-dihydroxyacetylphenone. In a similar manner (2-[(1E)-3-(2,4-dihydroxyphenyl)-3-oxoprop-1-en-1-yl]phenyl beta-D-glucopyranoside) can be generated starting from 2-hydroxybenzaldehyde and 2,4-dihydroxyacetylphenone. (2-[(1E)-3-(2,4-dihydroxyphenyl)-3-oxoprop-1-en-1-yl]phenyl beta-D-glucopyranoside) is the 2,2′,4′-trihydroxychalcone variant of isoliquiritin. The beta-D-glucopyranoside variant of a compound of the invention can be produced by an analogous method. The synthesis of 2,2′,4′-trihydroxychalcone is among others described in “Geissman T A and Clinton R O. Flavanones and related compounds; the preparation of polyhydroxychalcones and -flavanones. J Am Chem Soc. 1946 April; 68:697-700.”). Chalcones can also be synthesized by a classic Claisen-Schmidt condensation of a substituted acetophenone with a benzaldehyde derivative (Scheme 1 and Claisen L, et al, Ber deutch chem Ges 1881; 14:2460-2468 and Schmidt et al, Ber deutch chem Ges 1881; 14:1459-1461).

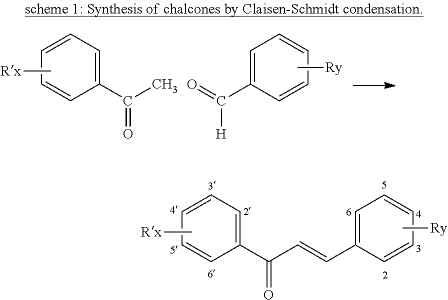

In one embodiment R1, R3 or R5 is a saccharide or an oligosaccharide. The oligosaccharide is preferably a disaccharide. The saccharide is preferably glucose or fructose, preferably D-glucose or D-fructose. The (oligo) saccharide is preferably linked to the chalcone backbone via an ether linkage on C1 of a six carbon saccharide and C2 of a five carbon saccharide. The disaccharide is preferably a 4-O-beta-D-apiofuranosyl(1-2)-beta-D-glucopyranosyl. In a preferred embodiment two of R1, R3, and R5 are OH and one of R1, R3, and R5 is a monosaccharide or oligosaccharide. In a preferred embodiment R1 and R3 are OH and R5 is a monosaccharide or oligosaccharide.

Inhibition of mTOR in a cell can be measured in various ways. Often this done by measuring the level of phospho-p70-S6K (pp70-S6K). The inhibition leaves preferably less than 90% of the mTOR activity intact when compared to the same conditions in the absence of a compound of formula I. The inhibition preferably leaves less than 80%, more preferably less than 70% more preferably less than 60%, 50% 40% and more preferably less than 20% of the mTOR activity intact when compared to the same conditions in the absence of a compound of formula I. The cell is preferably a cell that expresses mTOR.

Inhibition of IL-1β production by a cell is typically determined by means of an IL-1β specific ELISA. A suitable method for determining whether a compound inhibits the production is by measuring IL-1β expression in the medium of cells contacted with a compound of formula I. A suitable method for determining whether a compound inhibits the production is by measuring the level of mature IL-1β in the medium of cells contacted with a compound of formula I. The inhibition results in a reduction by at least 10% of the IL-1β levels produced when compared to the production of IL-1β under the same conditions in the absence of the compound of formula I. The inhibition is preferably a reduction by at least 20%, more preferably at least 30%, 40%, 50% and more preferably at least 70%, 80% and more preferably at least 90% of the IL-1β levels produced when compared to the production of IL-1β under the same conditions in the absence of the compound of formula I. The cell is preferably a cell that would otherwise (in the absence of the compound) produce IL-1β.

The level of IL-1β in blood is preferably measured by means of ELISA. The sample is preferably a plasma or serum sample of collected blood of an animal subject. IL-1B is preferably measured by means of high sensitivity ELISA. Preferably in a morning-fasting blood sample. The sample may be frozen after collection. A suitable ELISA kit can be obtained from BIOSOURCE, Camarillo, Calif.
Inhibition of IL-18 production by a cell is typically determined by means of an IL-18 specific ELISA. A suitable method for determining whether a compound inhibits the production is by measuring IL-18 expression in the medium of cells contacted with a compound of formula I. A suitable method for determining whether a compound inhibits the production is by measuring the level of mature IL-18 in the medium of cells contacted with a compound of formula I. The inhibition results in a reduction by at least 10% of the IL-18 levels produced when compared to the production of IL-18 under the same conditions in the absence of the compound of formula I. The inhibition is preferably a reduction by at least 20%, more preferably at least 30%, 40%, 50% and more preferably at least 70%, 80% and more preferably at least 90% of the IL-18 levels produced when compared to the production of IL-18 under the same conditions in the absence of the compound of formula I. The cell is preferably a cell that would otherwise (in the absence of the compound) produce IL-18.
The level of IL-18 in blood is preferably measured by means of ELISA. The sample is preferably a plasma or serum sample of collected blood of an animal subject. IL-18 is preferably measured by means of high sensitivity ELISA. Preferably in a morning-fasting blood sample. The sample may be frozen after collection. A suitable ELISA kit can be obtained from Quantikine HS, R&D systems, Minneapolis, Minn.

Inhibition of NRLP-3 is preferably measured by measuring inhibition IL-16 production. Inhibition of IL-1β production by a certain percentage is indicative for an inhibition NRLP-3 of the same percentage.

The invention further provides a method for inhibiting activation of caspase I by an inflammasome in a cell comprising providing a cell comprising said inflammasome with a compound of formula I. The invention further provides a method for the prevention of activation of a caspase I by an inflammasome, the method comprising providing a cell wherein is said inflammasome is to be activated, and contacting said cell with a compound of formula I. The inflammasome is preferably an NLRP-3 containing inflammasome. The cell is preferably a cell capable of expressing IL-1β and/or IL-18. The cell is preferably a cell capable of producing mature IL-1β and/or mature IL-18. The cell is preferably a myeloid cell, or mesenchymal stem cell. Inhibition of a caspase I containing inflammasome (preferably an NRLP-3 containing inflammasome) is preferably measured by the formation of caspase-1 from inactive caspase-I. Another method is a by measuring inhibition IL-1β production. Inhibition of caspase I formation from inactive caspase I by a certain percentage is indicative for an inhibition NRLP-3 containing inflammasomes of the same percentage. Inhibition of IL-1β production by a certain percentage is indicative for an inhibition NRLP-3 of the same percentage.

Stimulation of autophagy in a cell is preferably measured by detecting endogenous LC3 processing in the presence or absence of inhibitors of lysosomal turnover of autophagosome content. This is preferably complemented by analysis of effects of knockdown or knockout of autophagy regulators, such as Beclin-1 (for review see Barth et al 2010, J Pathol.; 221(2); 117-124 and specific references cited therein). Members of the LC3 family play a key role in the maturation of the autophagosome, the central organelle of autophagy. LC3 precursors are proteolytically processed to form LC3-I, which is diffusely distributed in the cytosol. Upon initiation of autophagy, the C-terminal glycine of LC3-I is modified by addition of a phosphatidylethanolamine (PE) to form LC3-II, which translocates rapidly to nascent autophagosomes in a punctate distribution. Therefore, lysosomal turnover of the autophagosomal marker LC3-II reflects autophagic activity, and detecting LC3 by immunoblotting or immunofluorescence has become a reliable method for monitoring autophagy and autophagy-related processes, including autophagic cell death.

Stimulation of phagocytosis is preferably measured by detecting ingested fluorescent particles by cells from the surrounding medium, such as fluorescent latex beads.

The mTOR is expressed in practically every cell of the body. In a method of inhibiting MTOR-expression of the invention, the cell is preferably a tumor cell, a cell of the central nervous system, preferably a neuron or a glial cell, preferably a microglial cell.

NLRP-3 is relevant for cell of the innate immune system and the immune system in the brain, Methods for inhibiting NLRP-3 of the invention are preferable methods involving cells of the innate immune system and immune cells of the brain. Preferred examples of such cells are a macrophage; a monocyte or a microglial cell.

Most cells are able of autophagy. A cell in a method of stimulating autophagy can therefore be any cell type. In a preferred embodiment the cell is a tumor cell, a cell of the central nervous system, preferably a neuron or a glial cell, preferably a microglial cell.

The cell in a method of inhibiting IL-1β production is preferably a macrophage; a monocyte or a microglial.

Like resveratrol and ILG, a compound of structural formula I, or at least the structural backbone that is substituted, can be obtained from a natural source.

An animal subject as indicated herein is said to have low grade inflammation, or is suffering from inflammaging when the plasma level of one or more inflammation cytokines is higher than normal for an average healthy young adult of approximately 20-30 years old. The low grade inflammation is chronic when the animal subject has said higher level or levels in two or more samples taken over a prolonged period such as for instance 6 months. An animal subject or subject as indicated herein is said to have low grade inflammation, or is suffering from inflammaging when the plasma level of IL16 is more than 0.05 pg/ml, preferably more than 0.1 pg/ml. An animal subject or subject as indicated herein is said to have low grade inflammation, or is suffering from inflammaging when the plasma level of IL18 is more than 150 pg/ml, preferably more than 300 pg/ml for men, or more than 120 pg/ml, preferably more than 240 pg/ml for women. An animal subject or subject as indicated herein is said to have low grade inflammation, or is suffering from inflammaging when the caspase-1 level in cells of the animal subject or subject is more than 40 pg/ml, preferably more than 80 pg/ml. The values for the plasma levels can be values for embodiments wherein the animal is a human.

A food or food supplement of the invention is typically provided with the compound of formula I. The food preferably comprises between 0.01 and 6% weight percentage of the compound of formula I based on the total weight of the food, preferably 0.02 and 4% of the compound of formula I (weight percentage based on the total weight of the food). In a preferred embodiment the food comprises 0.04 and 2% of the compound of formula I (weight percentage based on the total weight of the food). Preferably the food comprises at least 0.1% of the compound of formula I (weight percentage based on the total weight of the food). The food can be a solid, a fluid or a combination thereof. In a preferred embodiment the food comprises or is a dairy product, preferably a milk or fermented milk product, such as drinking yoghurt. In the presence invention a solution consisting essentially of water is not considered a food. Likewise, a watery solution for laboratory purposes is not a food or food supplement.

A food supplement typically comprises 20-2000 mg of the compound of formula I, preferably 40-1500 mg of the compound of formula I, preferably 80-1000 mg of the compound of formula I. The supplement is preferably provided in the form of a tablet, pill, or capsule. The tablet, pill, or capsule preferably comprises a coating to facilitate release of the compound from the formulation in the stomach or further in the gastrointestinal tract.

A pharmaceutical composition typically comprises 100-5000 mg of the compound of formula I, preferably 50-4000 mg of the compound of formula I, preferably 100-3000 mg of the compound of formula I and preferably 200-2000 mg of the compound of formula I. The pharmaceutical composition is preferably provided in the form of a tablet, pill, or capsule. The tablet, pill, or capsule preferably comprises a coating to facilitate release of the compound from the formulation in the stomach or further in the gastrointestinal tract.

The invention further provides a method of stimulating autophagy in a cell, the method comprising contacting the cell with a compound of structural formula I. Autophagy (or autophagocytosis) is a natural mechanism that disassembles, through a regulated process, unnecessary or dysfunctional cellular components. Autophagy allows the orderly degradation and recycling of cellular components. During this process, targeted cytoplasmic constituents are isolated from the rest of the cell within a double-membraned vesicle known as an autophagosome. The autophagosome then fuses with a lysosome and the contents are degraded and recycled. Three different forms of autophagy are commonly described: macroautophagy, microautophagy and chaperone-mediated autophagy. In the context of the present invention, autophagy can be been seen as an adaptive response to stress, which promotes survival. An increase in autophagy is preferably measured determining the intracellular level of conversion of LC3-I into LC3-II.

The invention further provides a method of treatment of an animal subject that has a disease associated with caspase-1, interleukin-1β and/or IL-18 over-expression, or has an increased risk of developing said disease the method comprising administering the compound of formula I to the animal subject in need thereof. Also provided is a compound of formula I for use in the treatment of an animal subject that has a disease associated with caspase-1, interleukin-1β and/or IL-18 over-expression, or has an increased risk of developing said disease. Diseases that are associated with IL-1β and/or IL-18 over-expression included but are not limited to multiple sclerosis, amyotrophic lateral sclerosis, gout, COPD, macular degeneration, hypertension, osteoporosis, osteoarthritis, rheumatoid arthritis, Duchenne muscular dystrophy, chronic fatigue syndrome (also referred to as myalgic encephalomyelitis), depression, non-fatty acid liver disease (NAFLD) and fibrosis. Preferred fibrosis are cardiac fibrosis, pulmonary fibrosis. In a preferred embodiment the disease is multiple sclerosis, amyotrophic lateral sclerosis, gout, COPD, macular degeneration, hypertension, osteoporosis, osteoarthritis, rheumatoid arthritis, Duchenne muscular dystrophy, non-fatty acid liver disease (NAFLD) and fibrosis. Preferred fibrosis are cardiac fibrosis, pulmonary fibrosis. In a preferred embodiment the disease is preferably pulmonary fibrosis. The disease is preferably a chronic disease with an auto-inflammatory component. A disease is said to have a chronic auto-inflammatory component if the pro-inflammatory cytokines IL-1β and/or IL-18 are dysregulated which is exemplified by them exceeding a threshold level in the plasma of the individual. Diseases that have an auto-inflammatory component are for instance rheumatoid arthritis, multiple sclerosis, Duchenne muscular dystrophies and other muscular dystrophies.

Also provided is the use of a compound of formula I for the preparation of a medicament; a food or a food supplement for the treatment of an animal subject that has a disease associated with caspase-1, interleukin-1β and/or IL-18 over-expression, or has an increased risk of developing said disease. Further provided is a compound of formula I for use in the treatment of a disease that benefits from inhibiting caspase-1, interleukin-1β and/or IL-18 production by a cell of said animal subject. Preferred diseases are multiple sclerosis, amyotrophic lateral sclerosis, gout, COPD, macular degeneration, hypertension, osteoporosis, osteoarthritis, rheumatoid arthritis, Duchenne muscular dystrophy, chronic fatigue syndrome (also referred to as myalgic encephalomyelitis), depression, non-fatty acid liver disease (NAFLD) and fibrosis. Preferred fibrosis are cardiac fibrosis, pulmonary fibrosis. Preferred diseases are multiple sclerosis, amyotrophic lateral sclerosis, gout, COPD, macular degeneration, hypertension, osteoporosis, osteoarthritis, rheumatoid arthritis, Duchenne muscular dystrophy, chronic fatigue syndrome (also referred to as myalgic encephalomyelitis), depression, non-fatty acid liver disease (NAFLD) and fibrosis. Preferred fibrosis are cardiac fibrosis, pulmonary fibrosis. The disease is preferably a chronic disease with an auto-inflammatory component. A disease is said to have a chronic auto-inflammatory component if the pro-inflammatory cytokines IL-1β and/or IL-18 are dysregulated which is exemplified by them exceeding a threshold level in the plasma of the individual. Diseases that have an auto-inflammatory component are for instance rheumatoid arthritis, multiple sclerosis, Duchenne muscular dystrophies and other muscular dystrophies.

Also provided is the use of a compound of formula I for the preparation of a medicament; a food or a food supplement for the treatment of an animal subject that has an increased risk of developing multiple sclerosis, amyotrophic lateral sclerosis, gout, COPD, macular degeneration, hypertension, osteoporosis, osteoarthritis, rheumatoid arthritis, chronic fatigue syndrome (also referred to as myalgic encephalomyelitis), depression, non-fatty acid liver disease (NAFLD) and fibrosis. Preferred fibrosis are cardiac fibrosis, pulmonary fibrosis. The disease is preferably a chronic disease with an auto-inflammatory component. An individual has an increased risk of developing said disease if it has LGI.

The invention also provides a method for the prevention and treatment of cerebral and ageing disorder in an animal subject the method comprising administering a compound of formula I to the animal subject in need thereof.

With a disease that benefits from treatment is meant that an animal subject with the disease benefits from the treatment, preferably by a symptom that is ameliorated or in that the life span is increased by the treatment.

Also provided is a compound of formula I for use in the treatment of an animal subject with a disease characterized in that symptoms of the disease are ameliorated by inhibiting a Mechanistic Target Of Rapamycin (mTOR) protein and/or NLRP-3 protein in a cell of said animal subject. Diseases of which symptoms of are ameliorated by inhibiting a Mechanistic Target Of Rapamycin (mTOR) protein and/or NLRP-3 protein in a cell of the animal subject are among others multiple sclerosis, amyotrophic lateral sclerosis, gout, COPD, macular degeneration, hypertension, osteoporosis, osteoarthritis, rheumatoid arthritis, chronic fatigue syndrome (also referred to as myalgic encephalomyelitis), depression, non-fatty acid liver disease (NAFLD) and fibrosis. Preferred fibrosis are cardiac fibrosis, pulmonary fibrosis. Preferred diseases are multiple sclerosis amyotrophic lateral sclerosis, gout, COPD, macular degeneration, hypertension, osteoporosis, osteoarthritis, rheumatoid arthritis, non-fatty acid liver disease (NAFLD) and fibrosis. Preferred fibrosis are cardiac fibrosis, pulmonary fibrosis.

Further provided is a compound of formula I for use in the treatment of an animal subject with a disease characterized in that symptoms of the disease are ameliorated by stimulating autophagy and/or phagocytosis in a cell of said animal subject. Diseases of which symptoms are ameliorated by stimulating autophagy and/or phagocytosis are multiple sclerosis, amyotrophic lateral sclerosis, gout, COPD, macular degeneration, hypertension, osteoporosis, osteoarthritis, rheumatoid arthritis, chronic fatigue syndrome (also referred to as myalgic encephalomyelitis), depression, non-fatty acid liver disease (NAFLD) and fibrosis. Preferred fibrosis are cardiac fibrosis, pulmonary fibrosis; Preferred diseases are multiple sclerosis, amyotrophic lateral sclerosis, gout, COPD, macular degeneration, hypertension, osteoporosis, osteoarthritis, rheumatoid arthritis, non-fatty acid liver disease (NAFLD) and fibrosis. Preferred fibrosis are cardiac fibrosis, pulmonary fibrosis.

Others have performed experiments with LDLR-deficient mice (a model for familial hypercholesterolemia). Such mice have been transplanted with bone marrow from NLRP3−/− mice. Mice whose bone marrow-derived cells lacked NLRP3 inflammasome components (or IL-1 cytokines) were markedly resistant to developing atherosclerosis.

### Hypertension

Others have performed experiments with wild-type and inflammasome-deficient ASC−/− mice. These mice were uninephrectomized and received deoxycorticosterone acetate and saline to drink (1K/DOCA/salt). 1K/DOCA/salt-induced hypertension in mice was associated with increased renal mRNA expression of inflammasome subunits NLRP3, ASC and pro-caspase-1, and the cytokine, pro-IL-16, as well as protein levels of active caspase-1 and mature IL-16. Following treatment with 1K/DOCA/salt, ASC−/− mice displayed blunted pressor responses and were also protected from increases in renal expression of IL-6, IL-17A, CCL2, ICAM-1 and VCAM-1, and accumulation of macrophages and collagen. Finally, treatment with a novel inflammasome inhibitor, MCC950, reversed hypertension in 1K/DOCA/salt-treated mice. Renal inflammation, fibrosis and elevated BP induced by 1K/DOCA/salt treatment are dependent on inflammasome activity, highlighting the inflammasome/IL-1β pathway as a therapeutic target in hypertension.

### Non Alcoholic Fatty Liver Disease (NAFLD)

Others have performed experiments with NLRP3 knockout mice. The mice were placed on short-term choline-deficient amino acid-defined (CDAA) diet, to induce isolated hepatic steatosis or long-term CDAA exposure, to induce severe steatohepatitis and fibrosis, respectively. Nlrp3−/− mice were protected from long-term feeding CDAA-induced hepatomegaly, liver injury, and infiltration of activated macrophages. More importantly, Nlrp3−/− mice showed marked protection from CDAA-induced liver fibrosis. In the liver samples of patients with NAFLD, inflammasome components were significantly increased in those patients with nonalcoholic steatohepatitis (NASH) when compared to those with non-NASH NAFLD with mRNA levels of pro-IL1 beta correlated to levels of COL1A1. The study uncovers a crucial role for the NLRP3 inflammasome in the development of NAFLD. These findings may lead to novel therapeutic strategies aimed at halting the progression of hepatic steatosis to the more severe forms of this disease.

### Duchenne Muscular Dystrophy.

Inflammation typically exacerbates disease progression in DMD patients. Suppression of the inflammation by steroids is rapidly becoming the standard of care for DMD patients. Boursereau et al found that downregulation of the NLRP3 inflammasome rescues Duchenne muscular dystrophy in a mouse model of DMD thereby identifying an auto-inflammatory component in disease progression of DMD (Boursereau et al BMC Biology (2018) doi.org/10.1186/s12915-018-0501). Inhibition of an auto-inflammatory component of the disease with a compound of the invention reduces the detrimental effect of the immune component and delay's disease progression in DMD patients and other muscular dystrophies.

### Ischemia Reperfusion Injury Prevented by Resveratrol

Others have performed experiments with myocardial ischemia/reperfusion animal models. The animals were induced by occlusion of the left anterior descending coronary arteries (LADs) for 30 min, followed by 2 h of reperfusion. Resveratrol was administered in different doses (2.5, 5, and 10 mg/kg) at the same time as the onset of reperfusion. The myocardial structure in myocardial ischemia reperfusion injury (MI/RI) rats was extensively damaged. After preconditioning with different concentrations of resveratrol (2.5, 5 and 10 mg/kg), the pathology and morphology were significantly improved in a dose-dependent manner. The results showed that resveratrol treatment significantly reduced the infarct volume and myocardial fibrosis, resulting in myocardial cells that lined up in a more orderly fashion and dose-dependent decreases in TnT and CK-MB levels in the serum of the I/R rats. Resveratrol also significantly modulated mRNA and protein levels by down-regulating NRLP3 and Caspasel expression and IL-1β and IL-18 activation. These results show that the NRLP3 inflammasome is activated during the myocardial I/R injury process and that the secretion of the inflammatory cytokines IL-1β and IL-18 mediates the cascade inflammatory response.

### Isoliquiritigenin has a Protective Effect on Cerebral Ischemia

Others have performed experiments with the protective effects of ILG. The effects were investigated in transient middle cerebral artery occlusion (MCAO)-induced focal cerebral ischemia-reperfusion injury in rats. ILG was administered once a day, for 7 days prior to ischemia. The rats were subjected to 2 h right MCAO via the intraluminal filament technique and 22 h reperfusion. Pretreatment with ILG significantly reduced the cerebral infarct volume and edema and produced significant reduction in neurological deficits. (Protective effects of isoliquiritigenin in transient middle cerebral artery occlusion-induced focal cerebral ischemia in rats.)

A compound of the invention is also useful in the treatment of astrogliosis, balance/coordination dysfunction and for the increasing bone mass is situations were such is desired, for instance in situations where bone mass decreases. Such situations may be osteoporosis, loss of bone mass as a result of prolonged inactivity such as hospitalization or aging in general. Others have performed experiments with aging wild type and NLRP3 knockout mice. Aged wild type mice spontaneously develop severe astrogliosis, functional decline and loss of bone mass. 24 month old NLRP3−/− mice show significant less astrogliosis and were protected from age-related decline in cognition and memory. The knockout mice performed significantly better in a Rotarod test, which measures balance and coordination and they could walk further than WT mice. Also 24 month old NLRP3−/− mice were protected from spontaneous, age related, loss of bone mass (Youm Y H et al. Cell Metab. 2013 Oct. 1; 18(4): 519-532)

### Longevity

Others have shown that rap amycin, an inhibitor of the mTOR pathway, extends median and maximal lifespan of both male and female mice when fed beginning at 600 days of age. Based on age at 90% mortality, rapamycin led to an increase of 14% for females and 9% for males. With increasing the longevity in a non-diseased animal subject is meant that the lifespan of the non-diseased animal subject is increased when compared to a control that is otherwise treated essentially the same but for the administration of the compound of formula I. The maximal lifespan can be increased, the median lifespan can be increased or both. With non-diseased animal subject is meant that the treatment is initiated at a time point when the animal subject is not known to have a disease. Such a state is typically also referred to as a healthy animal subject.

As used herein, the term “longevity” refers to the lifespan of the animal Thus, longevity refers to the number of years in the lifespan of an animal In some embodiments, the term “increased longevity” with regard to subjects administered the compositions of the invention, means that the lifespan of a non-diseased animal administered the compositions is increased relative to another non-diseased animal not administered the compositions. In some embodiments, the longevity of the animal is increased at least 6 months, at least 1 year, at least two years, at least 3 years, at least 4 years, at least 5 years, or at least 10 years compared to a non-diseased animal not administered the compositions. In some embodiments the longevity of the animal is increased at least one year, but not more than 10 years, not more than 5 years, or not more than 4 years compared to a non-diseased animal not administered the compositions.

The invention further provides a method for increasing the health span in a non-diseased animal subject, the method comprising administering to the animal subject an effective amount of a hydroxychalcone of formula I

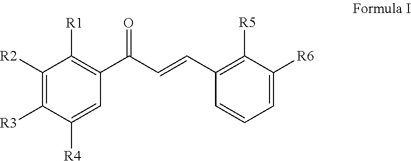

wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH. The animal subject is preferably a human. In a preferred embodiment R1=OH, R2=H, R3=OH, R4=H, R5=OH and R6=H.

A person's health span is the length of time that the person is healthy—not just alive. Treatment with a hydroxychalcone as described herein increases the length of time that a subject stays healthy or disease free when compared to subjects that are not thus treated. Without being bound by theory it is believed that lowering of an elevated blood interleukin-1β level, an elevated blood interleukin-18 level and/or the treatment of low grade inflammation in an animal subject in need thereof delays the onset of debilitating diseases such as multiple sclerosis, amyotrophic lateral sclerosis, gout, COPD, macular degeneration, hypertension, osteoporosis, osteoarthritis, rheumatoid arthritis, chronic fatigue syndrome (also referred to as myalgic encephalomyelitis), depression, non-fatty acid liver disease (NAFLD) and/or fibrosis. Preferred fibrosis are cardiac fibrosis, pulmonary fibrosis.

An animal subject is preferably a human subject, a farm animal or a pet animal. In a preferred embodiment the animal subject is a mammal, preferably a human.

A compound of formula I

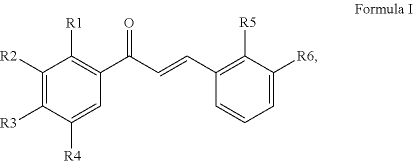

as a compound itself be it in a pharmaceutical, food or food supplement or the like, in any use or a method as disclosed herein, be it in a medical setting in a prophylactic/prevention setting or the like, is preferably a compound of formula I wherein R1=OH, R2=H, R3=OH, R4=H, R5=OH and R6=H. This preference is the same in any combination of features as disclosed herein.

For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1: IL-1β levels in LPS/ATP stimulated, and PMA differentiated THP-1 cells.

FIG. 2: IL-18 levels in LPS/ATP stimulated, and PMA differentiated THP-1 cells

FIG. 3: mTOR and autophagy activity in LPS/ATP stimulated and unstimulated PMA differentiated THP-1 cells.

FIG. 4: Caspase-1 level in LPS/ATP stimulated PMA differentiated THP-1 cells. The active form of caspase-1 can be measured in the supernatant of cells.

FIG. 5: The effect of 2,2′,4′-trihydroxychalcone treatment on the survival of nematodes.

FIG. 6: Table of suitable chalcones. The chalcones are numbered in order estimated activity. The chalcone with the highest expected activity received number 1. The one with the next highest expected activity number 2 etc.

FIG. 7: Estimated affinity, ligand efficiency (LE), Lipophilic ligand efficiency (LLE) and torsion angle (TOR) of several hydroxychalcone, compared to Glyburide, a known NLRP3 inhibitor. The drawing on the right shows the position of the 2,2′-dimethyl-4″-hydroxychalcone within the NLRP3 PYD domain.

Ligand Efficiency LE. The ligand efficiency is a measure for the activity normalized by the number of non-H atoms. More precisely, it is the relative free binding energy in kcal/mol per non-H atom, calculated from an IC50 value. Especially in early project stages prioritizing compounds based on their ligand efficiency values is a much more favorable approach compared to judging from plain activities alone: “For the purposes of HTS follow-up, we recommend considering optimizing the hits or leads with the highest ligand efficiencies rather than the most potent.” (Ref.: A. L. Hopkins et al., Drug Disc. Today, 9 (2004), pp. 430-431).

To give an example: A compound with 30 atoms (400 MW) that binds with a Kd=10 nM has a ligand efficiency value of 0.36 kcal/mol per non-H atom. Another compound with 38 non-H atoms (500 MW) and the same ligand efficiency would have a 100 fold higher activity with Kd=0.106 nM. Let us assume an HTS screening revealed two hit compounds A and B with equal activities of IC50=10 nm, but different molecular weights of 400 and 500, respectively. Based on activities both compounds look equally attractive. Considering, however, that a synthetic introduction of a new group with 8 non-H atoms into compound A would match compound B in terms of weight, but would increase the activity by a factor of 100, if its ligand efficiency value can be maintained, it becomes clear that compound A is the by far more attractive alternative.

### Lipophilic Ligand Efficiency LLE

The LLE value is a builds on the fact that the typical compounds of drug discovery projects huddle at the lipophilic side of the acceptable lipophilicity range. A gain in lipophilicity therefore is associated with a loss in bioavailability and should be compensated by higher activity on the target. To express this relationship the LLE is calculated as LLE=−log IC50−c Log P. A rough rule of thumb may be the suggestion of Jonathan S. Mason from Lundbeck Research to aim for LLE values above 3 for lead compounds and above 5 for clinical candidates.

### Torsion Angle (TOR)

Every molecule has a preferred (lowest energy) shape. The color coding indicates whether the shape of the molecule in the structure deviates from this lowest energy shape. Green indicates a good fit of the molecule when in it's preferred shape. Orange and red indicates an increasing deviation from the preferred fit. All indicated chalcones have a green symbol at TOR. For glyburide the symbol is red.

### EXAMPLES

### Example 1

### Determination of IL-1β, IL-18, Phospho P70 S6K and Caspase-1 Levels in THP-1 Cells.

Evaluation of the effect of test compounds on inflammasome induced IL-1β or IL-18 generation and mTOR or caspase-1 activity was performed using PMA-differentiated THP-1 cells stimulated with LPS (2 ng/ml, 4h (LPS (sigma, cat #L3012)) plus ATP (5 mM, 5 min. (Sigma, cat #A1852)). PMA-differentiated THP-1 cells untreated with LPS/ATP were used as control. PMA (phorbol 12-myristate 13-acetate) stimulation was done by adding 150 nM PMA to the cell culture for 24 hours. followed by 24 hours of incubation in culture medium and removal of non-adherent cells.

The supernatant was collected immediately after experimental procedures for measurement of IL-1β ELISA (R&D Systems, cat #DY201) or IL-18 ELISA (R&D Systems, cat #7620) levels.

The cell lysates were collected accordingly the manufacturer's instructions for analysis of phospho p70 S6K (Thr389) (eBioscience, cat #85-86052) levels by ELISA, to determine the mTOR activity or to determine caspase-1 (R&D Systems cat #AF6215) levels by Western blot. As secondary antibody for the Western blot a HRP-conjugated anti-IG antibody (R&D Systems cat #HAF017) was used and immunoreactive bands were visualized by the Enhanced Chemiluminescence method (Thermo Scientific, Rockford, Ill.). All assays were performed accordingly to the respective manufacturers instructions.

### Test Compounds

2,2′,4′-trihydroxychalcone (abcr cat #AB151762), isoliquiritigenin (TCI Europe cat #I0822) and resveratrol (TCI Europe cat #R0071). As control Rapamycin (APExBIO, cat #A8167) was used.

### Conditions Evaluated (LPS/ATP Unstimulated and LPS/ATP Stimulated Cells):

a) Dose response curves for test compounds: 5 points of 3-fold dilutions beginning from 300 μM
b) Control conditions (all contain comparable amount of DMSO, e.g. <1%)

### 1. Untreated

2. LPS+ATP only,

### 3. Rapamycin (25, 50 and 100 nM)

### Cellular Model:

PMA-differentiated THP-1 cells, 1.5×10\hat{ }5 cells/well, 96-well format

### Procedures: (in Order of Execution):

1. Pre-treatment for 1 hour with experimental compounds.
2. LPS stimulation (2 ng/mL) for 4h of all conditions except unstimulated (LPS/ATP unstimulated) cells
3. ATP (5 mM) stimulation for 5 minutes of all conditions except unstimulated (LPS/ATP unstimulated) cells
4. Material collection: Supernatant and cell lysates (snap frozen, stored at −80° C.)

### Results

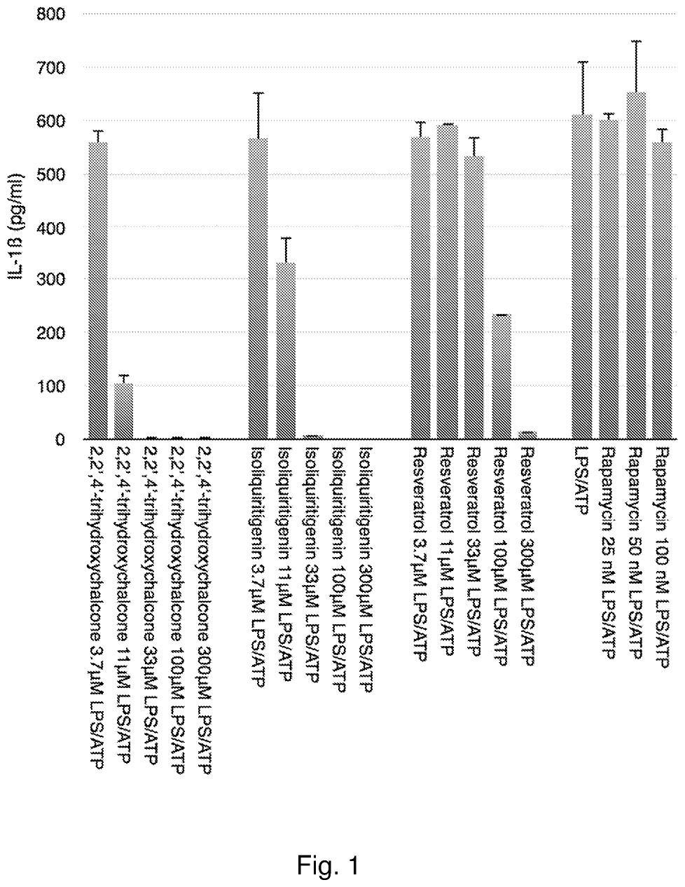

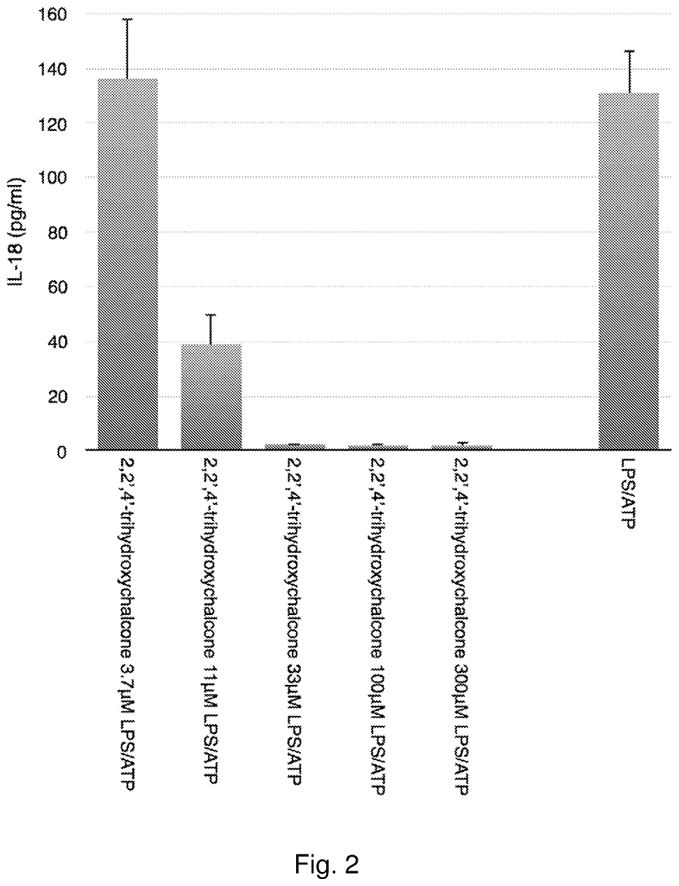

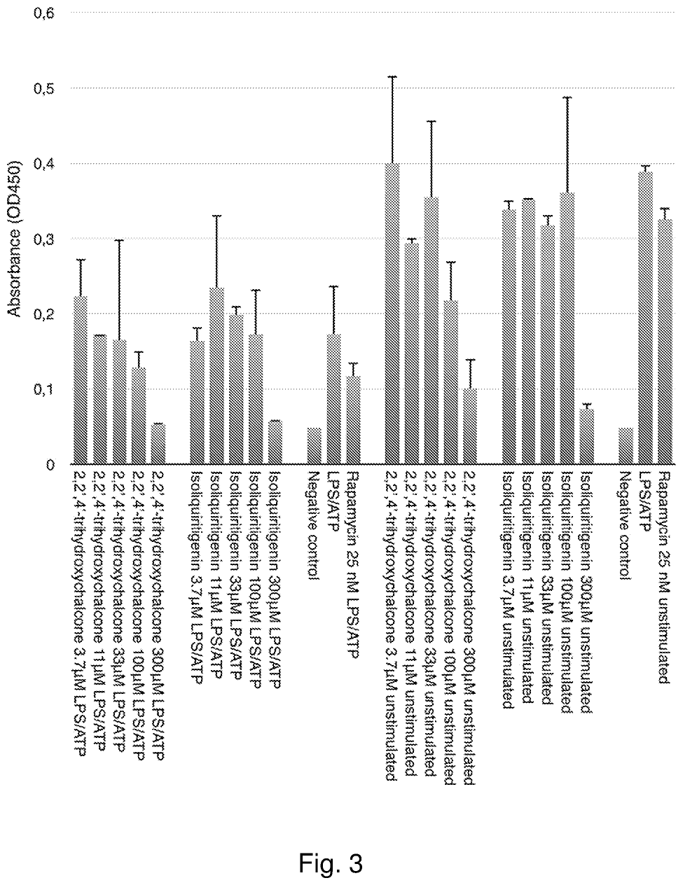

IL-1β secretion in LPS/ATP stimulated, PMA differentiated THP-1 cells.
FIG. 1 shows the results of an assay with PMA differentiated THP-1 cells which were LPS/ATP stimulated. It can be seen that the increasing concentrations of the test compounds: 2,2′,4′-trihydroxychalcone; Isoliquiritigenin; Resveratrol inhibited the production of IL-1β in these cells. Rapamycin had no effect.
The test compounds inhibit the secretion of IL-1β by PMA differentiated THP-1 cells, that are stimulated with LPS/ATP, in a dose dependent fashion. The IC50 of the tested compounds is:
6.1 μM for 2, 2′, 4′-trihydroxychalcone
11.9 μM for Isoliquiritigenin
87.3 μM for Resveratrol
   Since IL-1β secretion, under these conditions, is the result of activation of inflammasomes it can be concluded that the test compounds inhibit inflammasome induced IL-1β production. 2,2′,4′-trihydroxychalcone was the most effective inhibitor. Furthermore, since inhibition of mTOR by rapamycin did not inhibit inflammasome induced IL-1β production it can be concluded that these two processes can operate through independent mechanisms.
   Unstimulated PMA differentiated THP-1 cells did not produce IL-1β above the detection limit of the procedure (4 pg/ml).
   IL-18 secretion in LPS/ATP stimulated, PMA differentiated THP-1 cells
   FIG. 2 shows the results of an assay with PMA differentiated THP-1 cells which were LPS/ATP stimulated.
   2,2′,4′-trihydroxychalcone inhibits the secretion of IL-18 by PMA differentiated THP-1 cells, that are stimulated with LPS/ATP, in a dose dependent fashion. Since, under these conditions, IL-18 secretion is the result of activated inflammasomes, it can be concluded that 2,2′,4′-trihydroxychalcone inhibits inflammasome induced IL-18 production

Measurement of mTOR and autophagy activity by determination of the phosphorylation status of p70-56K in LPS/ATP stimulated and unstimulated PMA differentiated THP-1 cells.

The compounds 2,2′,4′-trihydroxychalcone; Isoliquiritigenin and rapamycin were added in various concentrations to LPS/ATP stimulated and unstimulated PMA-differentiated THP-1 cells.

The results show that the test compounds lead to dephosphorization the P70 S6K protein in a dose dependent fashion, both in PMA differentiated THP-1 cells, that are stimulated with LPS/ATP or in unstimulated PMA differentiated THP-1 cells. Rapamycin 25 nM was used as positive control. Inhibition in both unstimulated and stimulated cells is indicative for an increase in autophagy. Since inhibition was seen in both unstimulated and stimulated cells it is clear that mTOR regulated autophagy acts independently from the processes involved in inflammasome formation.

Since inhibition of mTOR leads to dephosphorization of the P70 S6K protein and subsequently to an upregulation of autophagy activity it can be concluded that the test compounds inhibit mTOR activity and upregulate autophagy. The IC50 of the test compounds in LPS/ATP stimulated and PMA differentiated THP-1 cells is found to be 98.8 μM for 2,2′,4′-trihydroxychalcone and 174.2 μM for Isoliquiritigenin The compound of formula I is thus a more active inhibitor of mTOR than Isoliquiritigenin.

### Determination of Caspase-1 Levels in LPS/ATP Stimulated, PMA Differentiated THP-1 Cells

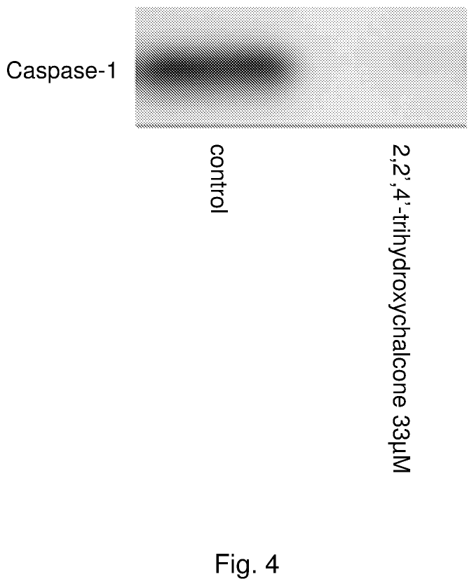

LPS/ATP stimulated, PMA differentiated THP-1 cells were incubated with a compound of formula I and caspase-I formation was analyzed and compared to a control without the compound. The result of a western blot for caspase-I is indicated in FIG. 4.

The results show that 2,2′,4′-trihydroxychalcone inhibits caspase-1 production in LPS/ATP stimulated, PMA differentiated THP-1 cells. Since, under these conditions, caspase-1 production is the result of activated inflammasomes, it can be concluded that 2,2′,4′-trihydroxychalcone inhibits inflammasome induced caspase-1 production.

### Example 2

### Materials and Methods

In this study the N2, bristol (wild-type) strain was used. Nematodes were maintained at 15° C. on nematode growth medium (NGM) seeded with Escherichia coli feeding strain 0P50.
For the treatment group, 2,2′,4′-trihydroxychalcone was dissolved in DMSO (final concentration 50 μM) and added to the NGM medium and the OP50 feeding suspension. For the control group, DMSO was added to the NGM medium and OP50 feeding suspension. Both the 2,2′,4′-trihydroxychalcone and the control plates contained a final DMSO concentration of 0.3% (v/v) during the whole experiment. For the aging assay, synchronous populations were obtained by allowing 5-10 hermaphrodites to lay eggs for 4 h. Lifespan scoring was initiated after hermaphrodites completed the final larval molt (day 1 of experiment). The starting number of nematodes was 100 per group. During the reproductive period, adult nematodes were transferred daily to new treatment plates to avoid overcrowding. Following post-reproduction, transfer occurred every third day, until the impact of aging disallowed handling of the nematodes. Survival was scored as the number of animals responsive to gentle touch as a fraction of the original number of animals on the plate.

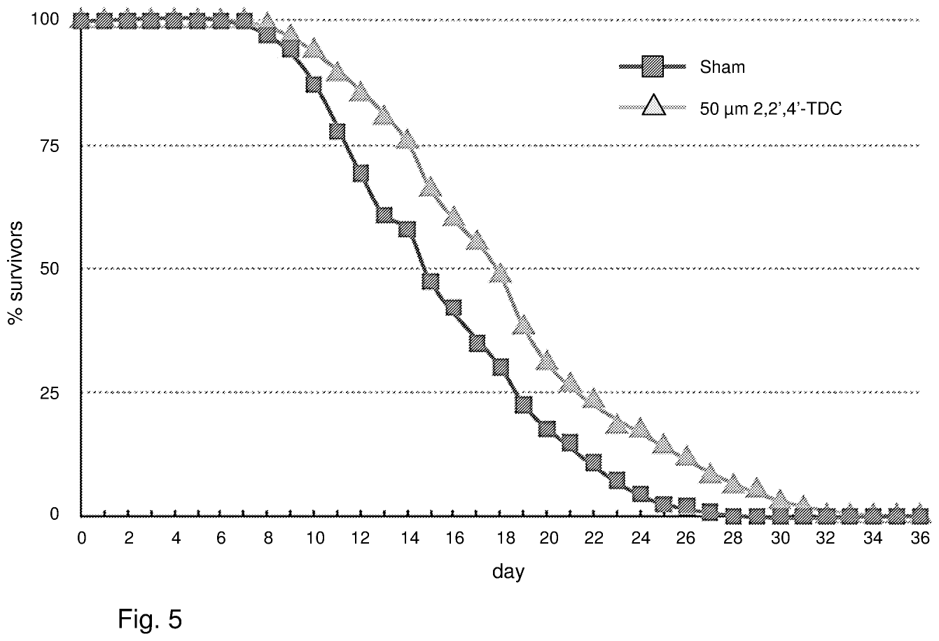

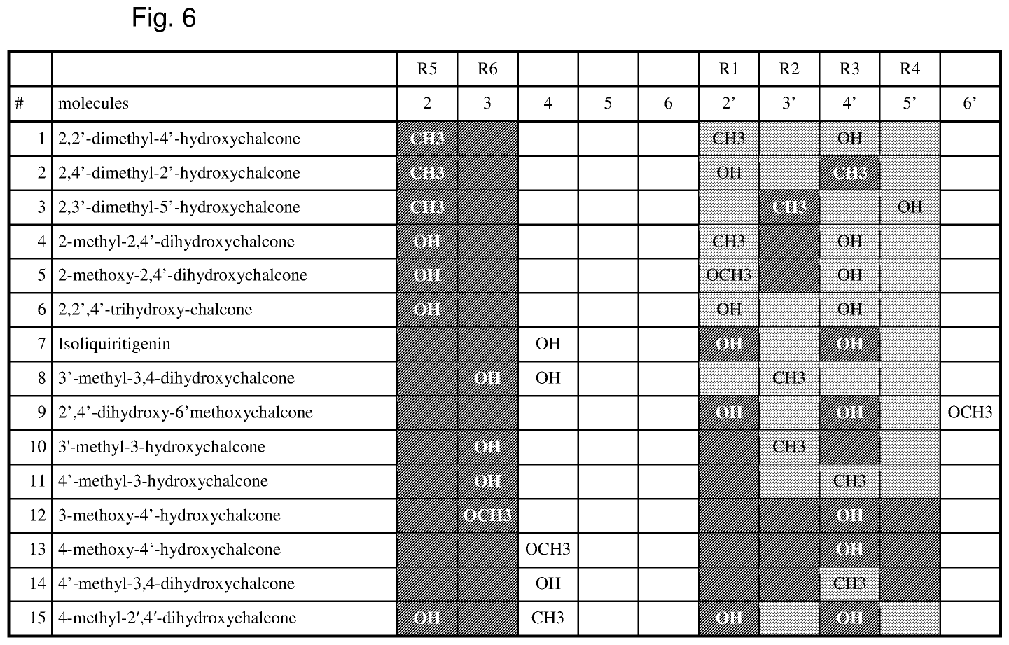

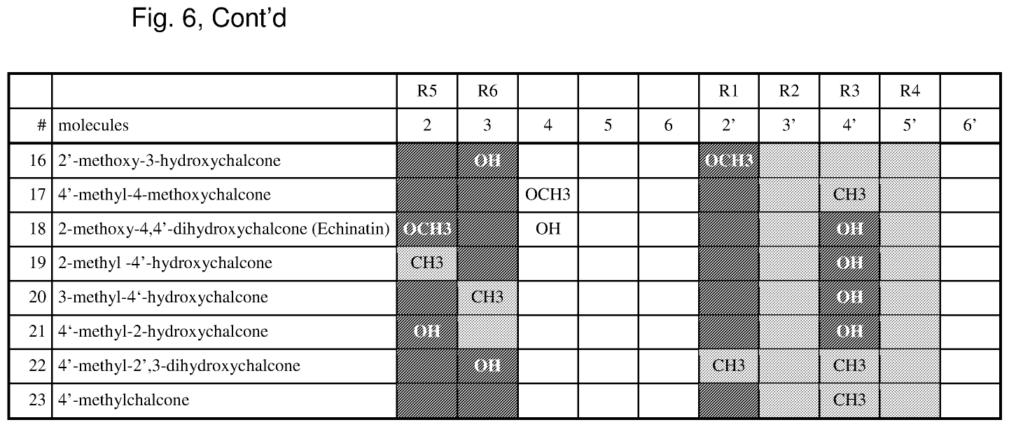

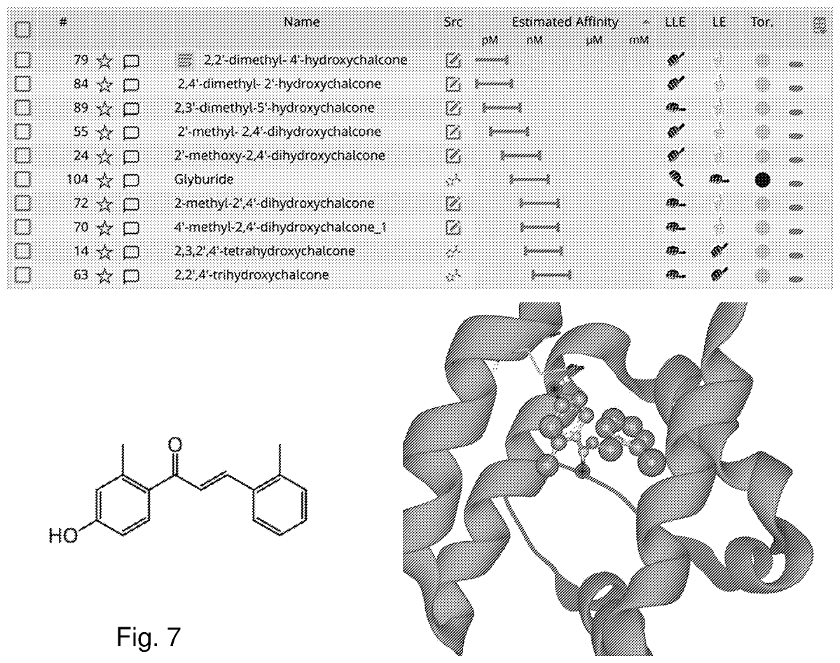

The results of the experiment are presented in FIG. 5.

### Example 3

Methods to formulate a chalcone as indicated herein.

### 1. Nano Particle Formulation

at least one emulsifier is dissolved in water. The emulsifier can for instance be a polysaccharide (gum ghatti), a lecithin, an ester of monoglyceride and a fatty acid, a mono- or diglycerides of a fatty acids.
2,2′,4′-trihydroxychalcone powder (crystals) are mixed into the solution. Subsequently an alcohol (for example glycerin or butanediol) is added to the emulsion. The size of the 2,2′,4′-trihydroxychalcone powder (crystals) in the emulsion is milled to on average <1 μm with a wet mill grinding (DYNO-MILL® KDL, Willy A Bachofen AG), high pressure dispersion or sonification. This will generate a stable nanosolution of 2,2′,4′-trihydroxychalcone, which contains 1-25% 2,2′,4′-trihydroxychalcone, 20-80% alcohol (glycerin) 0.5-15% emulsifier (gum ghatti) and 10-70% water.

### 2. Niosome Entrapment Formulation

Niosomes are nonionic surfactant-based vesicles with a similar structure to that of liposomes and can carry both hydrophilic and hydrophobic drugs within the same system.

### Example Preparation of Spray-Dried Niosomes

A surfactant mixture (Tween 80 and Span 80, (ratio (mol/mol) 1:0.01-0.01:1) and cholesterol (ratio (mol/mol) 1:0.5-1:1) was dissolved in 80 mL of methanol/dichloromethane (ratio 4:1-2:1, v/v). The solvent was evaporated at 37° C. under vacuum by a rotary evaporator. The resulting dried film was redissolved in 60 mL of ethyl ether, and a solution containing 2,2′,4′-trihydroxychalcone in a mixture of dehydrated alcohol and phosphate-buffered solution (pH 6.5) was then added (ratio 2,2′,4′-trihydroxychalcone: carriers (w/w) 4:100-25:100) Next, 20 mL of phosphate-buffered solution was added after 10 minutes of sonication. Rotary evaporation was performed again at 60° C. until hydration was achieved and the residual ethyl ether was removed. The niosomal suspension was left to mature overnight at 30° C. in a thermostatic water bath shaker. In order to obtain a uniform particle size, the niosomal suspension was homogenized using a high pressure homogenizer

### Preparation of Niosomal Powder

Powder-derived niosomes are superior to conventional niosomes in terms of convenience of storage, transport, and dosing. To prevent degradation, fusion, and leakage, the niosomal powder was prepared using spray-drying method. Mannitol was added into the niosomal suspension as a protectant to prevent drug leakage upon dehydration using each drying method. Six grams of mannitol was added into 100 mL of niosomal suspension before the drying method was performed.

### Spray-Drying Method

The spray-drying process was done using a spray-dryer. The niosomal suspension was fed to the spray chamber by a pump. The aspirator setting, airflow rate, inlet temperature, and speed of the pump were kept at the scale of 100%, 357 L/hour, 130° C., and 1.5 mL/minute, respectively. Finally, the resulting powder was separated from the hot air stream with cyclone and collected in the bottom of the chamber.

### Food Product

2,2′,4′-trihydroxychalcone is added to a dairy product, for example milk, yoghurt or butter. Final concentration between 0.01 and 2.5%.
2,2′,4′-trihydroxychalcone is added to a beverage for instance tea, coffee, a herbal drink or an energy drink
   The 2,2′,4′-trihydroxychalcone is preferably added as a freeze dried powder.
   Hydroxychalcone (nano) particles are preferably provided with a coating that dissolves after ingestion. In other words a coating that dissolves, disintegrates and/or disrupts after the material has been swallowed. The coating is preferably a gel coating or an enteric coating.

1. A method for the prophylactic or curative treatment of an elevated blood interleukin-1β level, an elevated level of IL-18 and/or the treatment of low grade inflammation in an animal subject comprising administering a hydroxychalcone of formula I 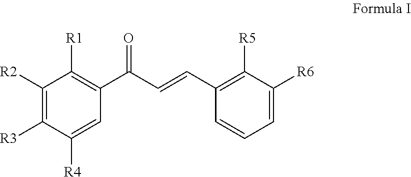
wherein R1=OH, R2=H, R3=OH, R4=H, R5=OH and R6=H; to animal subject in need thereof.

2. The method of claim 1, wherein the elevated blood interleukin-1β level, an elevated level of IL-18 and/or the treatment of low grade inflammation is caused by a disease selected from the group consisting of multiple sclerosis, amyotrophic lateral sclerosis, gout, COPD, macular degeneration, hypertension, osteoporosis, osteoarthritis, rheumatoid arthritis, Duchenne muscular dystrophy, chronic fatigue syndrome (also referred to as myalgic encephalomyelitis), depression, non-fatty acid liver disease (NAFLD) and/or fibrosis.

3-4. (canceled)

5. A method of inhibiting caspase-1, interleukin-1β and/or IL-18 production by a cell, the method comprising contacting the cell with a hydroxychalcone of formula I 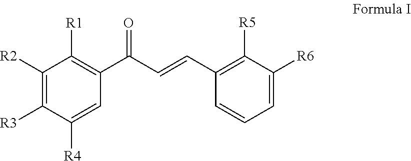
wherein R1=OH, R2=H, R3=OH, R4=H, R5=OH and R6=H.

6.-8. (canceled)

9. A method for increasing longevity and/or increasing health span in a non-diseased animal subject, the method comprising administering to the animal subject an effective amount of a hydroxychalcone of formula I 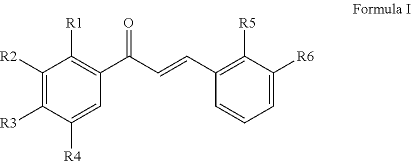
wherein R1, R2, R3, R4, R5 and R6 are each independently H, OH, CH3, OCH3, a monosaccharide, an oligosaccharide or Cl; with the proviso that at least two of R1-R6 are H; and at least one other of R1-R6 is OH.

10. The method of claim 9, wherein
R1 is CH₃; R2 is H; R3 is OH; R4 is H; R5 is CH₃; R6 is H; or
R1 is OH; R2 is H; R3 is CH₃; R4 is H; R5 is CH₃; R6 is H; or
R1 is H; R2 is CH₃; R3 is H; R4 is OH; R5 is CH₃; R6 is H; or
R1 is CH₃; R2 is H; R3 is OH; R4 is H; R5 is OH; R6 is H; or
R1 is OCH3; R2 is H; R3 is OH; R4 is H; R5 is OH; R6 is H; or
R1 is OH; R2 is H; R3 is OH; R4 is H; R5 is CH₃; R6 is H; or
R1 is OH; R2 is H; R3 is OH; R4 is H; R5 is OH; R6 is OH; or
R1 is OH; R2 is H; R3 is OH; R4 is H; R5 is OH; R6 is H.

11. The method of claim 10, wherein R1=OH, R2=H, R3=OH, R4=H, R5=OH and R6=H.

12. The method of claim 9, wherein the animal subject is a human.

13-15. (canceled)
